(19) United States
(12) Patent Application Publication
(10) Pub. No.: US 2017/0028407 A1
(43) Pub. Date: Feb. 2, 2017

(54) AIR TREATMENT DEVICE

(71) Applicants: Gary Lee Fairbank, JR., Sherwood, MD (US); Dylan Ray Fairbank, Sherwood, MD (US)

(72) Inventors: Gary Lee Fairbank, JR., Sherwood, MD (US); Dylan Ray Fairbank, Sherwood, MD (US)

(21) Appl. No.: 15/226,507

(22) Filed: Aug. 2, 2016

Related U.S. Application Data
(63) Continuation-in-part of application No. 14/489,378, filed on Sep. 17, 2014, now abandoned.
(60) Provisional application No. 61/958,001, filed on Jul. 17, 2013.

(51) Int. Cl. B03C 3/019; A61L 9/14; B03C 3/155; B01D 46/00; B03C 3/60

(52) U.S. Cl. CPC B03C 3/019; B01D 46/0032; B01D 46/0028; B03C 3/60; B03C 3/155; A61L 9/14; B01D 2267/40; B01D 2279/65; A61L 2209/134; A61L 2209/211

(57) ABSTRACT

An air treatment device is provided. The air treatment device includes a negative air machine and a cold air fogger. The negative air machine includes an intake and an output for airflow. The cold air fogger may include a tank for including the fogging liquid and a delivery nozzle directed towards the output of the airflow of the negative air machine. Therefore, infected air may travel through the negative air machine and out of the output where the airflow is treated by the cold air fogger. The present invention may treat air within contaminated rooms.

### CROSS-REFERENCE TO RELATED APPLICATION

This application is a continuation-in-part of U.S. patent application Ser. No. 14/489,378, filed Sep. 17, 2014, which claims the benefit of U.S. Provisional Patent Application No. 61/958,001, filed Jul. 17, 2013, the contents of which are herein incorporated by reference.

### FIELD OF THE INVENTION

The present invention relates to air treatment device, and more particularly, to a negative air machine combined with an air filtration device and a cold air fogger to sanitize or disinfect air in an enclosed space, e.g., a room.

### BACKGROUND OF THE INVENTION

Air disinfectants are typically chemical substances capable of disinfecting microorganisms suspended in the air. Although glycols are effective air disinfectants in controlled laboratory environments, it is more difficult to use them effectively in real-world environments because the disinfection of aft is sensitive to continuous action. Mold intrusions are usually treated by spraying with bleach or contained through demolition. Current mold remediation is timely and therefore costly. The same can be said about attempting to disinfect an area affected with airborne viral particles.

As can be seen, there is a need for an improved air filtration devices to treat infected indoor areas.

### SUMMARY OF THE INVENTION

One aspect of the present invention provides an air treatment device containing a negative air machine for filtering the air through an air filter (such as a high-efficiency particulate air (HEPA) filter) and a cold air fogger. The negative air machine contains an intake and an output for air. The air filter is placed between the intake and output. The cold air fogger contains a tank and a delivery nozzle, wherein the tank contains hydrogen peroxide (H₂O₂) and silver. The delivery nozzle may preferably be directed towards the output of the negative air machine.

In another aspect of the present invention, the negative air machine also contains an electrostatic screen upstream of the air filter. The electrostatic screen may be made of a metal mesh screen that is electrically connected to a direct current source. In an embodiment, the electrostatic filtration screen is coated with a mixture containing activated carbon and silver.

A further aspect of the present invention provides a method for making the electrostatic screen. The method involves coating a metal mesh screen with an adhesive. A suspension containing activated carbon and silver is then coated over the adhesive. The twice coated metal mesh screen is then allowed to dry.

A yet further aspect of the present invention provides a modular air treatment device containing a frame to which the negative air machine and the cold air fogger may be removably attached thereto. The modular air treatment device allows for quick assembly and disassembly of the components of the air treatment device for ease of transportation.

These and other features, aspects and advantages of the present invention will become better understood with reference to the following drawings, description and claims.

### BRIEF DESCRIPTION OF THE DRAWINGS

FIG. 1 is a front perspective view of an embodiment of the present invention;

FIG. 2 is a back perspective view of an embodiment of the present invention;

FIG. 3 is an exploded view of an embodiment of the present invention;

FIG. 4 is an exploded view of the air dispersion assembly of FIG. 1;

FIG. 5 is a front view of an embodiment of the present invention;

FIG. 6 is a side view of an embodiment of the present invention;

FIG. 7 is an elevated view of the electrostatic screen; and

FIG. 8 is an elevated view of the electrostatic screen having an electrically insulated gasket thereon.

### DETAILED DESCRIPTION OF THE PREFERRED EMBODIMENTS

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an air treatment device for sanitizing or disinfecting air in an enclosed space. The air treatment device includes a negative air machine, preferably a HEPA negative air machine, and a cold air fogger. The negative air machine includes an intake and an output for airflow. In certain embodiments, an electrostatic screen may be placed in the negative air machine upstream of the air filter. The cold air fogger may include a tank for containing the fogging liquid with an adjustable electrostatic charge and a delivery nozzle directed towards the output of the airflow of the negative air machine. Therefore, infected air may travel through the negative air machine and out of the output where the airflow is treated by the cold air fogger. The present invention may treat air within any contaminated indoor areas and the present invention may easily disassemble and assemble within minutes for storage and transportation.

The present invention includes a machine comprised of a combination of environmental systems which delivers laboratory quality indoor air. The machine removes fungal and viral particles from within the air as well salmonella, MERSA and other biological organisms. The machine utilizes a combination of a negative air machine, a dry fog comprised of hydrogen peroxide and silver ions with adjustable electrostatic charge as well as UV bacteria disinfection light to provide clean indoor air. The present invention can be set in an affected area, and in less than four hours, the air will be clean and microorganisms on surfaces and in the air is sanitized or disinfected.

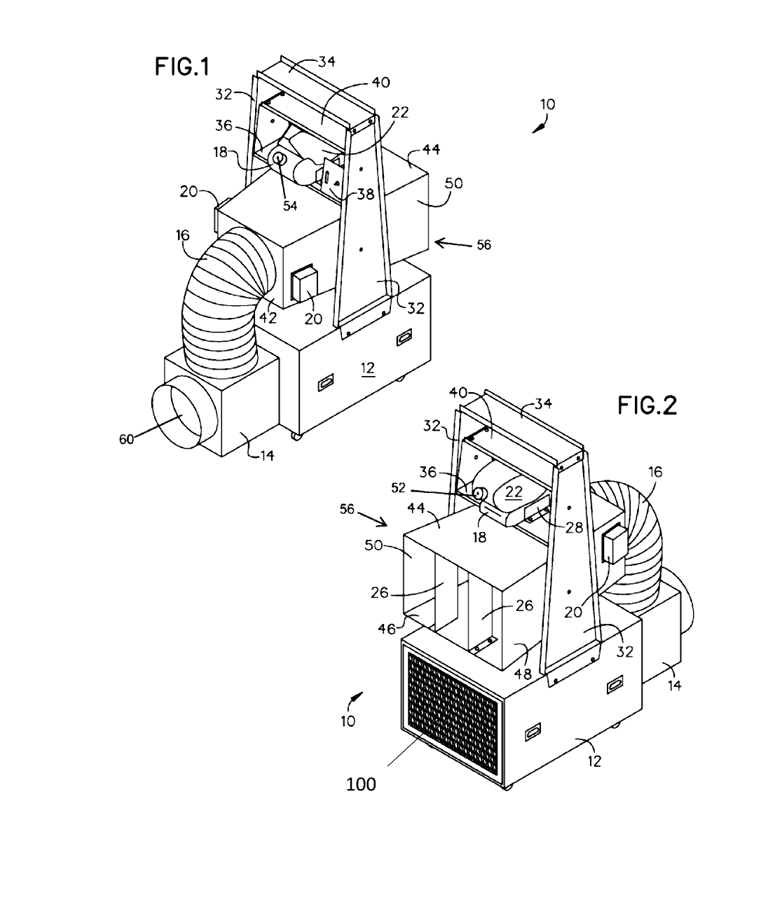

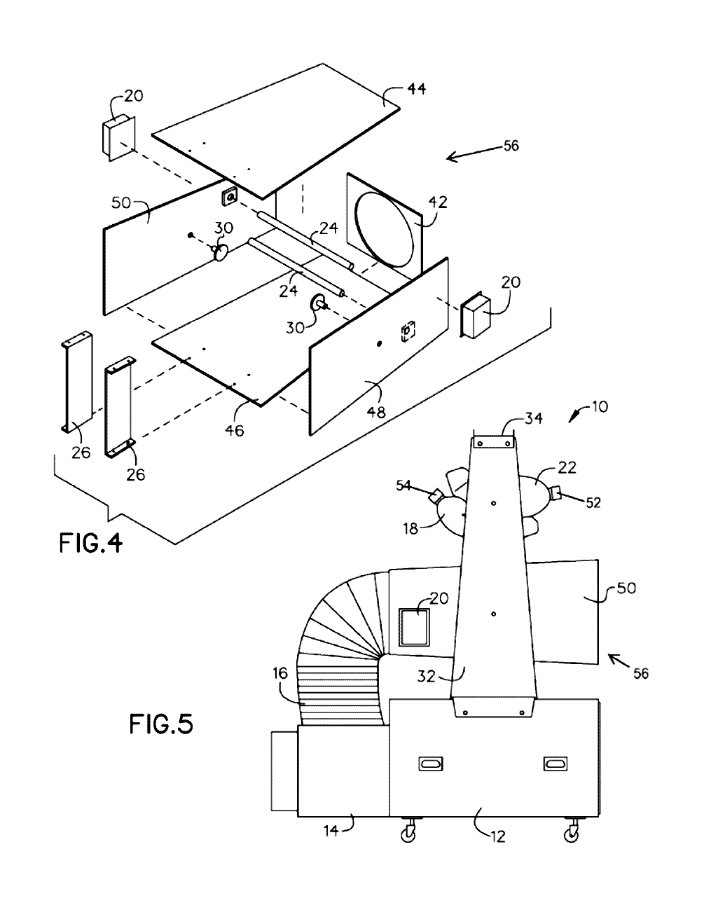

Referring to FIGS. 1 through 6, the present invention includes an air treatment device 10. The air treatment device 10 includes a combination of a negative air machine 12 and a cold air fogger 22. The negative air machine 12 may include an air filter, such as a high-efficiency particulate air (HEPA) filter, custom or high activated carbon filters for odor control therein. The negative air machine 12 further includes an intake and an output for air. Preferably, the negative air machine 12 is capable of moving a minimum of 2000 cubic feet per minute.

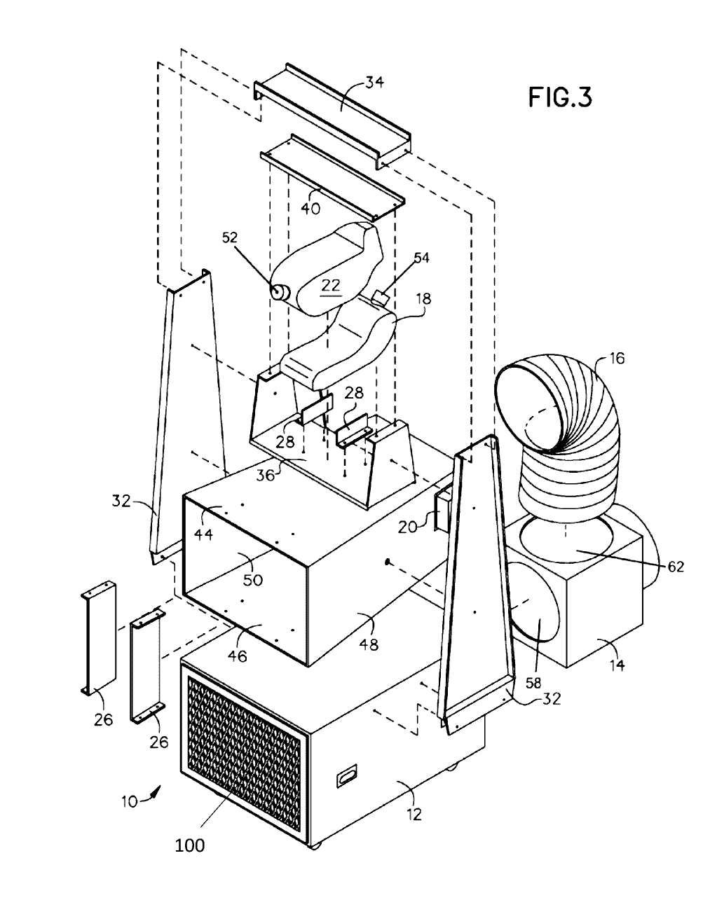

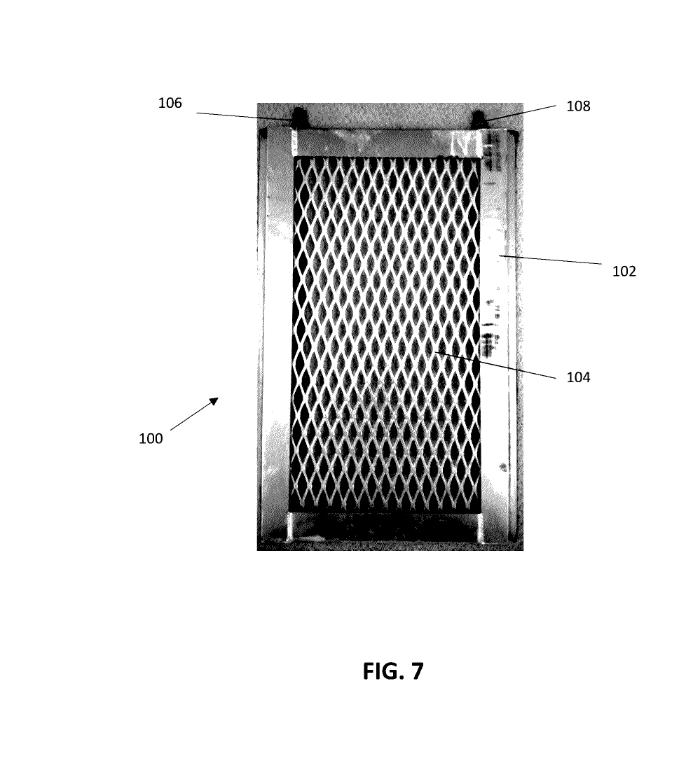

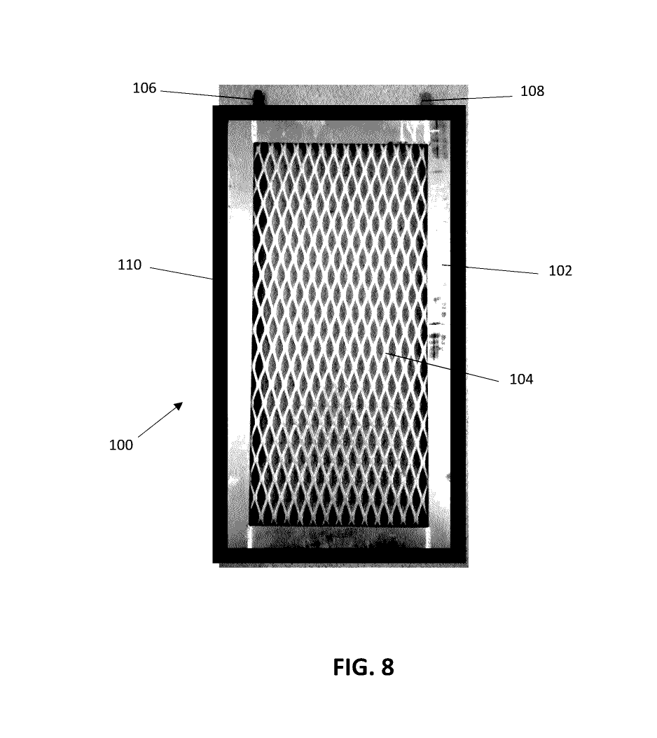

In certain embodiments, an electrostatic screen 100 may be added to the negative air machine 12 upstream of the air filter. Preferably, the electrostatic screen 100 is located at the intake of the negative air machine 12, as best shown in FIGS. 2-3 and 6. As best shown in FIG. 7, the electrostatic screen 100 contains a metallic mesh screen 104 that is attached to a metallic frame 102 around its perimeter. The frame 102 contains electrical connections 106, 108 for connecting the electrostatic screen 100 to a DC electrical source. The metallic mesh screen 104 preferably has a sieve opening of about 2 to about 4 mm. In a preferred embodiment, the frame is provided with an electrical insulator 110, such as a rubber gasket, around its perimeter, as best shown in FIG. 8, for electrical insulating the electrostatic screen 100 from the negative air machine 12 and the remaining air treatment device 10, when the electrostatic screen 100 is installed in place. Although FIG. 8 shown the electrical insulator 110 around the frame, other methods for electrically insulating the electrostatic screen 100 from the remaining air treatment device 10 may also be appropriate. For example, the intake of the negative air machine 12 may contain a recess for accepting and retaining the electrostatic screen 100, which may be lined with an electrical insulator. When installed, the electrical connections 106, 108 of the electrostatic screen 100 may be connected to a DC source for providing an electrical current to the electrostatic screen 100. The DC source is preferably an inverter located on a frame of the air treatment device 10 and is adjustable for providing a potential of about 110 to about 1V to the electrostatic screen 100.

In an embodiment, the electrostatic screen 100 contains a coating thereon, preferably on the metallic mesh screen 104. The coating contains a mixture containing activated carbon and silver. The coating, in dry state, contains about 92 to about 98% (by weight of the coating) carbon with the remaining being silver. To apply the coating on to the electrostatic screen 100, a water based suspension is provided which contains activated carbon and silver. The suspension contains about 40 to about 50% (by weigh of the suspension) water, about 46 to about 59% activated carbon, and about 1 to about 5% silver. Prior to loading the suspension on to the metallic mesh screen 104, the metallic mesh screen 104 surface is first prepared with an adhesive to promote adhesion of the activated carbon and silver to the metallic mesh screen 104. The adhesive is preferably a latex based adhesive which is sprayed on to the mesh screen 104. Other adhesives that is not affect by electrical current passing through the mesh screen 104 may also be appropriate for the present invention. The suspension is then placed on to the adhesive treated metallic mesh screen 104 and allowed to dry. To load the activated carbon and silver on to the mesh screen 104, the electrostatic screen 100 (with the adhesive treated mesh screen 104) is dipped into the suspension, so that it is completely covered. It is important to note that the metallic mesh screen 104, with the coating on it, should not be clogged or prevent air flow therethrough when in use with the negative air machine 12. The coating, when dried, should be no more than about 1 to about 2 mm thick on the metallic mesh screen 104, without clogging the mesh screen 104.

The cold air fogger 22 may include a tank 18 and a delivery nozzle 52. The tank 18 contains an aqueous mixture of hydrogen peroxide and silver ions and includes a cap 54 which may be removed to fill the tank 18 with the mixture. The cold air fogging machine 22 may include an adjustable droplet output size of about 10 to 40 microns and a dispersal rate of about 0.5 to 2 gallons per hour. An DC electrical source is operable to charge the mixture in the tank 18, which amplifies the effects of the disinfecting fog. The DC electrical source may be the same as that used for the electrostatic screen 100 and preferably is adjustable to provide a potential of about 1V to about 110V. Alternatively, the DC electrical source for the cold air fogger 22 may be a separate one than that for the electrostatic screen 100. An electrostatic charge may be activated by a power source and may be adjustable for fine tuning the fogging system. Preferably, the electrostatic charge is delivered to the nozzle of the fogger at less than about 5V. The present invention may disperse ionized silver which causes induction of H₂O₂ to mitochondria or bacteria. The cold air fogger 22 of the present invention dispenses the disinfecting fog towards the output of air from the negative air machine 12.

The aqueous mixture used by the cold air fogger 22 contains about 2 to about 8% (by weight of the mixture) hydrogen peroxide, preferably about 4-5%, and about 0.01 to about 0.05% (by weight of the mixture) silver, preferably about 0.01 to about 0.03%. In certain embodiments, the mixture may also contain an inorganic acid as a stabilizer.

The silver used in the suspension or the cold air fogger 12 may include, but not limited to, silver compounds include silver nitrate (AgNO₃), silver sulfate (Ag₂SO₄), silver chloride (AgCl), and the sodium/silver chloride complex having the formula AgNaCl₂. Additional suitable silver compounds are silver benzoate (C₆H₅—CO₂Ag), silver carbonate (Ag₂CO₃), silver fluoride (AgF), silver (I) oxide (Ag₂O) and silver (II] oxide (AgO). Colloidal silver compounds may also be used.

In certain embodiments, the present invention may include an air dispersion assembly 56. The air dispersion assembly 56 may be connected to the output of negative air machine 12 and may be positioned to blow air in an opposite direction of the intake of air of the negative air machine 12. The air dispersion assembly 56 may include an intake panel 42 include an opening, and a top panel 44, a bottom panel 46, a first side panel 48 and a second side panel 50 forming a channel within.

The present invention may further include a frame. The frame may mount the air dispersion assembly 56 above the negative air machine 12 and the cold air fogger 22 above the air dispersion assembly 56. As illustrated in the Figures, the frame may include side support beams 32 connected at the top by a top support beam 34. Below the top support beam 34 may be the middle shelf 40 and the sprayer shelf 36. The cold air fogger 18 may be secured in between the middle shelf 40 and the sprayer shelf 36 by supports 28. An adjustable electrostatic power source may run to the fogging fluid storage tank with an on/off switch delivered to the fogging mixture. Further, an on/off switch 38 may be secured to the sprayer shelf 36. The on and off switch 38 may be used to turn the air filtration device 10 on and off

The air dispersion assembly 56 may be mounted to the frame below the sprayer shelf 36 and above the negative air machine 12. In certain embodiments, the air dispersion assembly 56 may be pivotally mounted to the supports 32 by swivel pins 30. A directional air flow panel 43 is mounted to the air dispersion assembly 56 in order to pivot the air dispersion assembly 56 and support the air dispersion assembly 56 in the pivoted direction.

The present invention may further include a vent box 14 and a hose 16. The vent box may include an intake opening 58, a delivery opening 62, and an exhaust opening 60. The intake opening 58 is connected to the output of the negative air machine and the hose 16 connects the delivery opening 62 to the intake panel 42 of air dispersion assembly 56. The layout described above achieves a separation of positive and negative air flow for the purpose of filtering out particulate including biological and viral contaminants. In certain embodiments, a 4 inch opening is located in between the hose 16 and the air dispersion assembly 56 to allow for an even greater degree of concentrated air flow which allows for removal of contagions in smaller, hard to reach areas.

The exhaust opening 60 of the vent box 14 may be used to utilize the negative air machine 12 alone. Therefore, one of the exhaust opening 60 and the delivery opening 62 are closed during operation of the air filtration device 10.

The inside of the air dispersion assembly 56 may include additional components. For example, at least one Ultra violet (UV) lamp 24 may be suspended within the air dispersion assembly 56. In certain embodiments, two UV lamps 24 are installed inside of the air dispersion assembly 56. The UV lamps work in conjunction with the cold air fogger 22 to provide multiple levels of protection as these lights kill many types of viral/fungal contaminates. Preferably, the UV lamp 24 is disposed downstream of the air filter and the electrostatic screen 100.

Further, in certain embodiments the present invention may include a plurality of directional vents 26 mounted within the air dispersion assembly 56.

The present invention may further include a dehumidifier or a building dryer. The dehumidifier may be placed at one end of the present invention to quicken the disinfection process by aiding in the drying of the vapor dispelled by the cold air fogger.

The cold air fogger 22 may spray a disinfection solution that contains a mixture of hydrogen peroxide (H₂O₂) and silver ions. When the solution comes into contact with any biological/viral pathogen the silver ion serves to punch a hole in the cellular membrane of the contaminant. The hydrogen peroxide then infiltrates the cell through this pathway and reacts with the processes within the mitochondria shutting down energy production within the contaminant and effectively rendering the pathogen dead. The electrostatic charge delivered to the fogging mixture from the DC source serves to enhance the ability of the silver ions to permeate cell membranes. After about 90 minutes the hydrogen peroxide reacts with atmospheric oxygen to form oxygen and water. Therefore, the entire process is clean, green and completely harmless. The HEPA negative air machine serves to filter the air of all harmful particulates as well as influencing the directionality of the disinfecting fog to wherever the user desires. The attached ultraviolet disinfection light also provides additional protection as it kills certain fungal/viral particles upon contact.

The machine is simple to operate. A user may simply place the machine in the center of the affected area, fill it with the appropriate amount of disinfection liquid, set it to operate for the appropriate time (both determined by the size of the area and level of disinfection required) and leave the room. Returning is possible in two (2) hours as soon as the hydrogen peroxide oxygenates turning H₂O₂ into H₂O with a free oxygen molecule which will not raise oxygen levels in the area with the small amount of solution used in a room. At this time the indoor air is disinfected and the machine can be removed.

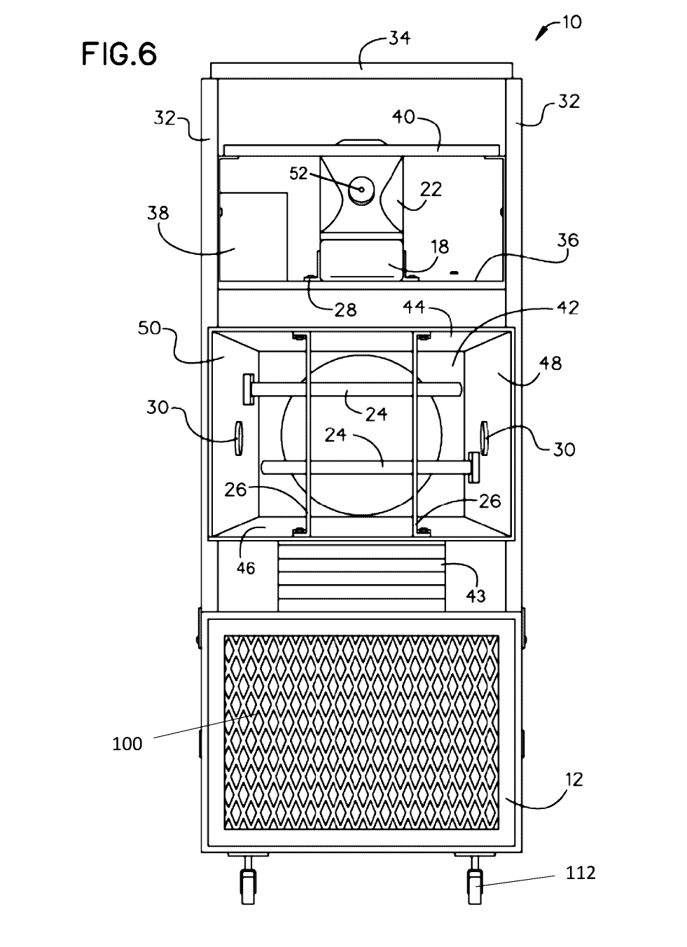

In an embodiment, the air treatment device 10 is modular and contains a frame to which the negative air machine and the cold air fogger may be removably attached thereto. The modular air treatment device 10 allows for quick assembly and disassembly of the components of the air treatment device for ease of transportation. In a preferred embodiment, the air treatment device 10 contains the following modular units: the air fogger 22, the air dispersion assembly 56, and the negative air machine 12. All three units are attached to the frame and/or each other via quick connect clips or connectors. The air fogger 22 may be contained in its own padded case and connects to its DC electrical source via electrical clips and slides in between the supports 28. The air dispersion assembly 56 may be connected to the side support beams 32 of the frame by a nut on each side. The negative air machine 12 may be connected to the bottom of the side support beams 32 via nuts, preferably two on each side. Instead of nuts, the air dispersion assembly 56 and the negative air machine 12 may be detachably connected to the frame, e.g., by releasable latches, clamps, or clips to allow the component to be quickly connected to and released from the frame. The bottom of the negative air machine 12 may contain wheels 112, as best shown in FIG. 6, for easy transport of the negative air machine 12, when disassembled, and of the air treatment device 10, when assembled. Disassembly of the unit merely requiring unclipping of the air fogger 22 from its DC electrical source and sliding it out of the supports 28, unclipping the electrostatic screen 100 from its DC electrical source, and removing the nuts holding the air dispersion assembly 56 and the negative air machine 12 to the frame. Assembly and disassembly of the air treatment device 10 may be accomplished in under 10 minutes.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is

1. An air treatment device comprising:
a. a negative air machine having an air filter within, wherein the negative air machine comprises an intake and an output for air;
b. an electrostatic screen upstream of the air filter; and
c. a cold air fogger comprising a tank and a delivery nozzle, wherein the tank contains a mixture of hydrogen peroxide and silver.

2. The device of claim 1, wherein the filter is a high-efficiency particulate (HEPA) filter.

3. The device of claim 1, the tank is electrically connected to a DC electrical source to electrically charge the mixture.

4. The device of claim 1, wherein the electrostatic screen is electrically connected to a DC electrical source to electrically charge the electrostatic screen.

5. The device of claim 1, wherein the electrostatic screen is located at the intake.

6. The device of claim 1, further comprising an air dispersion assembly connected to the output and positioned to blow air in the opposite direction of the intake air.

7. The device of claim 6, further comprising a vent box and a hose, wherein the vent box comprises an intake opening and a delivery opening, wherein the intake opening is connected to the output and the hose connects the delivery opening to the air dispersion assembly.

8. The device of claim 7, wherein the vent box further comprises an exhaust opening, wherein one of the exhaust opening and the delivery opening are closed during operation of the air filtration device.

9. The device of claim 6, wherein the negative air machine, the air dispersion assembly, and the cold air fogger are removably mounted to a frame.

10. The device of claim 6, further comprising at least one Ultra violet lamp suspended within the air dispersion assembly.

11. The device of claim 6, further comprising a plurality of directional vents mounted within the air dispersion assembly.

12. The device of claim 1, wherein the electrostatic screen contains a coating mixture comprising activated carbon, hydrogen peroxide, and silver.

13. An electrostatic screen comprising
a. a metallic mesh screen;
b. a metallic frame attached to a perimeter of the mesh screen; and
c. electrical connections on the frame,
wherein the mesh screen is coated with a mixture containing activated carbon, hydrogen peroxide, and silver.

14. A method for making an electrostatic screen comprising the steps of
a. providing the electrostatic screen containing a metallic mesh screen and a metallic frame attached to a perimeter of the mesh screen;
b. coating the mesh screen with an adhesive to provide and adhesive coated mesh screen; and
c. coating the adhesive coated mesh screen with a suspension comprising activated carbon and silver.

15. The method of claim 14, further comprising a step of drying the suspension.
